(19) United States
(12) Patent Application Publication
(10) Pub. No.: US 2025/0128083 A1
(43) Pub. Date: Apr. 24, 2025

(54) METHOD AND APPARATUS FOR TREATING REFRACTIVE ERROR OF THE EYE

(71) Applicant: ACUCELA INC., Seattle, WA (US)

(72) Inventors: Ryo KUBOTA, Seattle, WA (US); Amitava GUPTA, Seattle, WA (US)

(73) Assignee: ACUCELA INC., Seattle, WA (US)

(21) Appl. No.: 18/972,217

(22) Filed: Dec. 6, 2024

Related U.S. Application Data
(63) Continuation of application No. 17/052,906, filed on Nov. 4, 2020, now Pat. No. 12,226,647.
(60) Provisional application No. 62/669,580, filed on May 10, 2018.

(51) Int. Cl. A61N 5/06; A61N 5/067; G02C 7/04

(52) U.S. Cl. CPC A61N 5/0613; G02C 7/04; A61N 2005/0632; A61N 2005/0648; A61N 2005/0651; A61N 2005/0663; A61N 5/067; G02C 2202/24

(57) ABSTRACT

A source of light energy such as a source of violet light energy is coupled to a structure configured to contact the eye. The light source and structure are arranged to provide therapeutic amounts of violet light energy to the eye in order to inhibit the progression of refractive error such as myopia. The light source can be configured in many ways and may comprise a radioisotope and a phosphorescent material. The structure configured to contact the eye may comprise a contact lens or an implant.

CROSS REFERENCE

This application is a continuation of U.S. patent application Ser. No. 17/052,906, filed Nov. 4, 2020, which is a 371 national phase of PCT/US2019/030682, filed May 3, 2019, published as WO/2019/217241 on Nov. 14, 2019, and claims the benefit under 35 U.S.C. § 119 (e) of U.S. Provisional Application No. 62/669,580, filed May 10, 2018, the disclosures of which are incorporated, in their entirety, by this reference.

### BACKGROUND

Prior approaches to vision correction of refractive error of the eye are less than ideal in at least some respects. Although spectacles and contact lenses can correct vision, these corrective devices may not decrease the onset and severity of myopia and other refractive errors. Similarly, most surgical approaches do not address the underlying causes of ocular development that can result in the eye having refractive error.

In light of the above, improved methods and apparatus of treating refractive error of the eye are needed. Ideally, such methods and apparatus would at least partially address the progression and onset of refractive error of the eye such as myopia.

### SUMMARY

Embodiments of the present disclosure provide improved methods and apparatus for the treatment of refractive error with light, such as violet light. In some embodiments, a source of light energy such as a source of violet light energy is coupled to a structure configured to contact the eye. The light source and structure are arranged to provide therapeutic amounts of light such as violet light energy to the eye in order to inhibit the progression or onset of refractive error such as myopia. The light source can be configured in many ways and may comprise a radioisotope and a phosphorescent material. The structure configured to contact the eye may comprise a contact lens or an implant.

In a first aspect, an apparatus to treat refractive error of an eye comprises a structure to contact the eye, and a light source coupled to the structure. The light source is configured to direct light energy toward a retina of the eye to treat the refractive error of the eye.

In some embodiments, the light source emits violet light.

In some embodiments, the structure comprises a contact lens, and optionally the light source is one or more of embedded in the contact lens, located on an anterior surface of the contact lens, or located on a posterior surface of the contact lens.

In some embodiments, the light energy comprises violet light energy comprising a wavelength within a range from about 360 nm to about 400 nm.

In some embodiments, the light energy comprises violet light energy and the light source is configured to direct the violet light energy to the retina with an irradiance within a range from about 0.1 mW/cm2 to 5 mW/cm2.

In some embodiments, the light source illuminates a pupil of the eye with light energy in the range 0.1 nit to 10 nits and optionally within the range from 0.5 nits to 10 nits.

In some embodiments, the contact lens comprises one or more of a structure anterior to the light source to reflect light to the retina of the eye, a structure posterior to the light source to focus light onto the retina of the eye, a lens structure posterior to the light source to focus light onto the retina of the eye, or a diffractive structure posterior to the light source to diffract light toward the retina of the eye.

In some embodiments, the contact lens comprises a lens body, the lens body comprising one or more of a soft contact lens, a hydrogel contact lens, a hard contact lens, a rigid gas permeable contact lens, a polymethyl methacrylate contact lens, or an orthokeratology contact lens.

In some embodiments, the light energy comprises light energy violet light energy and the contact lens is configured to direct the violet light energy onto a cornea and toward the retina with an amount sufficient to promote a curvature change to the cornea of the eye.

In some embodiments, the structure comprises a contact lens comprising a posterior surface comprising a posterior radius of curvature sized to fit a cornea of the eye, and an anterior surface comprising an anterior radius of curvature configured to correct vision of the eye, and optionally the anterior surface comprises a second anterior radius of curvature oriented in relation to the anterior radius of curvature to correct an astigmatism of the eye.

In some embodiments, the structure to contact the eye comprises an implantable structure and the implantable structure comprises a covering disposed over the light source, and optionally the implant is configured to be turned on and off by a person who received the implant.

In some embodiments, the light source comprises one or more of a radioluminescent light source, a light emitting diode, a laser diode, a radioactive material, or a phosphorescent material, and optionally the radioactive material comprises tritium or radium.

In some embodiments, the structure to contact the eye comprises an optically transmissive material comprising a transmittance of at least 40% at 360 nm.

In some embodiments, the light source is arranged in a pattern on the structure to contact the eye, the pattern comprising one or more of: a spatial pattern on the structure to contact the eye, a spatial pattern located on an optically used portion of a contact lens, a spatial pattern on an inner portion of the contact lens to transmit light to the retina when the pupil constricts, a circular pattern, or a radial pattern, and optionally the structure to contact the eye comprises a contact lens.

In some embodiments, the apparatus comprises a contact lens comprising a posterior surface shaped to receive a cornea of the eye, a contact lens material comprising an index of refraction configured to transmit violet light from the source toward the retina, the contact lens comprising an anterior surface shaped to correct a refractive error of the eye in combination with the index of refraction and the posterior surface, and optionally the contact lens is configured to correct higher order aberrations comprising optical aberrations above third order, and optionally the contact lens comprises a multifocal contact lens configured to correct presbyopia.

In another aspect, a method comprises treating an eye with the apparatus of any one of the preceding claims.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a contact lens configured to treat refractive error of the eye in a cross-sectional view, in accordance with some embodiments.

FIG. 1B shows a front view of the contact lens of FIG. 1A.

### DETAILED DESCRIPTION

The methods and apparatus disclosed herein are well suited for combination with prior approaches to vision correction. For example, the structure to contact the eye may comprise a contact lens or an implant. Such structure may function normally to correct refractive errors and focus light into the eye, while the light source coupled to the structure may emit light such as violet light toward a retina of the eye to treat the refractive error of the eye. In some embodiments, such emission of violet light and violet light received by the eye may be controlled at a level so that there may not be noticeable influence on the functions of the eye.

In some embodiments, the apparatuses and methods disclosed herein are configured to prevent progression or onset of refractive errors in the eye(s). In some embodiments, the apparatuses and methods disclosed herein are configured to treat or improve refractive conditions in the eye(s). In some embodiments, the apparatuses and methods herein promote a curvature change to the cornea of the eye thereby improving refractive conditions in the eye(s), for example with orthokeratology assisted by light such as violet light. In some embodiments, the apparatuses and methods herein inhibit inappropriate increases or allow controlled amounts of increase to the axial length of the eye thereby reducing refractive errors in the eye(s) such as myopia.

In some embodiments, an apparatus to treat refractive error of an eye comprises: a structure to contact the eye; a light source coupled to the structure; wherein the light source is configured to emit violet light energy toward a retina of the eye to treat the refractive error of the eye. In some embodiments, the structure comprises a contact lens. The light source can be embedded in the contact lens, on an anterior surface of the contact lens, or on a posterior surface of the contact lens and combinations thereof. In some embodiments, the violet light comprises a wavelength within a range from about 360 nm to about 400 nm. In some embodiments, the light source is configured to direct violet light energy to the retina with a luminance is less than 5 mW/cm². In some embodiments, the light source illuminates the pupil of the eye with light energy in the range from 0.1 nit to 10 nit (candelas per square meter), preferably from 0.5 nit to 2 nits. In some embodiments, the contact lens comprises a structure anterior to the light source to reflect light to the retina of the eye, a structure posterior to the light source to focus light onto the retina of the eye, a lens structure posterior to the light source to focus light onto the retina of the eye, or a diffractive structure posterior to the light source to diffract light toward the retina of the eye, and combinations thereof. In some embodiments, the contact lens comprises a lens body, in which the lens body comprises a soft contact lens, a hydrogel contact lens, a hard contact lens, a rigid gas permeable contact lens, a polymethyl methacrylate contact lens, or an orthokeratology contact lens, and combinations thereof. In some embodiments, the contact lens is configured to direct the violet light energy onto a cornea and toward the retina with an amount sufficient to promote a curvature change to the cornea of the eye. In some embodiments, the structure comprises a contact lens comprising a posterior surface comprising a posterior radius of curvature sized to fit a cornea of the eye, and an anterior surface comprising an anterior radius of curvature configured to correct vision of the eye. In some embodiments, the anterior surface comprises a second anterior radius of curvature oriented in relation to the anterior radius of curvature to correct an astigmatism of the eye.

In some embodiments, the structure to contact the eye comprises an implantable structure, in which the implantable structure comprises a covering disposed over the light source. The implant can be configured to be turned on and off by a person who received the implant.

The light source can be configured in many ways. In some embodiments, the light source comprises a radioluminescent light source, a light emitting diode, a laser diode, a radioactive material, or a phosphorescent material and combinations thereof. The radioactive material may comprise tritium or radium, for example.

In some embodiments, the structure to contact the eye comprises an optically transmissive material comprising a transmittance of at least 40% at 360 nm.

In some embodiments, the light source is arranged in a spatial pattern on the structure to contact the eye. The spatial pattern can be located on an optically used portion of a contact lens, on an inner portion of the contact lens to transmit light to the retina when the pupil constricts, or in a circular pattern or a radial pattern on the contact lens, and combinations thereof.

In some embodiments, the apparatus comprises a contact lens comprising a posterior surface shaped to receive a cornea of the eye, a contact lens material comprising an index of refraction and configured to transmit violet light from the source toward the retina, an anterior surface shaped to correct a refractive error of the eye in combination with the index of refraction and the posterior surface. In some embodiments, the contact lens is configured to correct higher order aberrations comprising optical aberrations above third order, and the contact lens may comprise a multifocal contact lens configured to correct presbyopia.

In some embodiments, a method of treating the eye comprises treating an eye with the apparatus(es) as disclosed herein, such as a contact lens or an implant as disclosed herein.

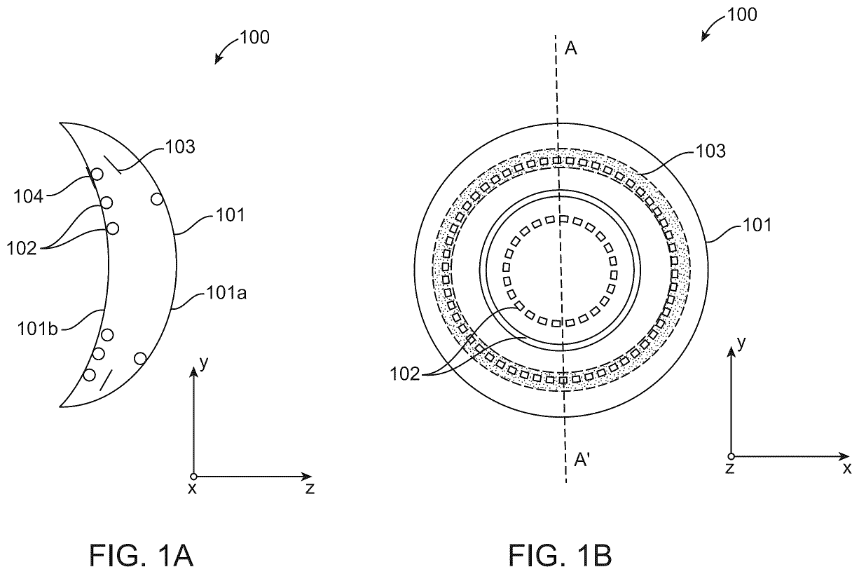

Referring to FIGS. 1A to 1B, in accordance with some embodiments, the apparatus 100 comprises a structure to contact the eye 101. In some embodiments, the structure 101 comprises a contact lens or an intraocular lens (IOL). In some embodiments, the contact lens 101 includes a lens body. Nonlimiting examples of the lens body include a soft contact lens, a hydrogel contact lens, a hard contact lens, a rigid gas permeable contact lens, a polymethyl methacrylate contact lens, or an orthokeratology contact lens, and combinations thereof. In some embodiments, the contact lens has a posterior surface 101b comprising a posterior radius of curvature sized to fit a cornea of the eye, and an anterior surface 101a comprising an anterior radius of curvature configured to correct vision of the eye. In some embodiments, the anterior surface also includes a second anterior radius of curvature oriented in relation to the anterior radius of curvature to correct astigmatism of the eye. In some embodiments, the contact lens material has an index of refraction and is configured to focus violet light from the source toward the retina. In some embodiments, the anterior surface 101a is shaped to correct a refractive error of the eye (e.g., myopia, hyperopia, presbyopia, and astigmatism) with a combination of the index of refraction and the posterior surface 101b. In some embodiments, the contact lens is configured to correct higher order aberrations comprising optical aberrations of third order, e.g. coma, and above, e.g. spherical aberration. In some embodiments, the contact lens comprises a multifocal contact lens configured to correct presbyopia.

In some embodiments, the contact lens material has an index of refraction and is configured to transmit violet light from the source toward the retina. In some embodiments, the structure to contact the eye 101 has an optically transmissive material comprising a transmittance of at least 30%, 40%, 50%, or 60% at one or more of 350 nm, 360 nm, 370 nm, 380 nm, 390 nm, or 400 nm. In some embodiments, the transmittance is at least 30%, 40%, 50%, or 60% in the entire range from 350 nm to 400 nm.

In some embodiments, the apparatus 100 includes the light source 102 that is coupled to the structure 101. The light source 102 may comprise a plurality of light sources. In some embodiments, the light source is embedded in the structure, and the light source may be embedded in one or more locations of the structure. Such locations may include the optically used portion of the lens. For example, the light source can be located anywhere on an anterior surface 101a of the contact lens 101, or on a posterior surface 101b of the contact lens and combinations thereof. In some embodiments, the light source may be spatially distributed in various patterns. In some embodiments, the spatial pattern may be located on an optically used portion of a contact lens, on an inner portion of the contact lens, or both, to transmit light to the retina (e.g., when the pupil constricts). In some embodiments, the light source may be distributed or in a circular pattern or a radial pattern on the contact lens, and combinations thereof. As shown in FIG. 1B, the light source may be distributed in multiple concentric rings with fixed or variable gaps therebetween. In some embodiments, the light source may be distributed in a donut pattern. Nonlimiting examples of patterns in which the light source may be spatially distributed include: a web-like pattern, a spiral pattern, a swirl pattern, and a radially scatter pattern (optionally from the center of the structure). In some embodiments, the spatial pattern may include areas that are at or substantially at the circumference of the structure (e.g., less than 3 mm, 2 mm, 1 mm, 0.5 mm, 0.2 mm or less to the circumference). As an example, the light source includes multiple concentric rings with a largest ring covering the entire circumference.

In some embodiments, the light source is configured to emit optical energy toward a retina of the eye to treat the refractive error of the eye. As shown in FIGS. 1A and 1B, the light source emits violet light toward the eye. FIG. 1A shows the cross section at A-A′ in FIG. 1B. The violet light may comprise a wavelength within a range from about 350 nm to about 400 nm. In some embodiments, the light source is configured to direct violet light energy to the retina with a radiance less than 5 mW/cm². The radiance of the violet light can be within a range from about 0.01 mW/cm² to about 5 mW/cm², for example. Continuing to refer to FIGS. 1A-1B, in some embodiments, the contact lens 101 has a structure 103 anterior to the light source to reflect light to the retina of the eye. Alternatively or in combination, a structure 104 can be located posterior to the light source 102 in order to focus light onto the retina of the eye. The structure 104 may comprise a lens structure posterior to the light source to focus light onto the retina of the eye, or a diffractive structure posterior to the light source to diffract light toward the retina of the eye, and combinations thereof. Alternatively or in combination, the light source can be located closer to the posterior surface of the lens than the anterior surface in order to decrease absorbance of the contact lens material.

In some embodiments, the structure to contact the eye 101 comprises an implantable structure which may comprise a covering disposed over the light source and optionally wherein the implant is configured to be turned on and off, for example, by a person who received the implant or by a trigger external to the implant such as intensity of ambient light detected by the implant.

In some embodiments, the contact lens 101 or the implant is configured to direct the violet light energy onto a cornea and toward the retina with an amount sufficient to promote a curvature change to the cornea of the eye. In some embodiments, the light source is configured to emit the light at a substantially constant level (e.g. within 25%) for a pre-determined period of time, e.g., for at least 12 hours, 1 day, 1 week, 2 weeks, 1 month, or even longer. In some embodiments, the light source is configured to emit the light when triggered by an external trigger. Non-limiting exemplary triggers may include ambient light, opening or closing of the eyelid, a temperature at the light source, etc. In some embodiments, the light source is configured to remain substantially fixed in its spatial location relative to the eye of the patient.

In some embodiments, the light source 102 is configured to direct violet light energy to the retina with a luminance. The light source may have a radioluminescent light source, a light emitting diode, a laser diode, a radioactive material, a phosphorescent material, or a chemiluminescent compound, and combinations thereof. In some embodiments, the radioactive material includes tritium and/or radium.

In some embodiments, the irradiance (energy per unit area) of the violet light (e.g., having a wavelength within the range of 350 nm to 400 nm) emitted from the light source and/or that reaches the eye is not particularly limited. In some embodiments, the irradiance is preferably determined by taking into consideration the effect of the on the human eye and skin, and the duration of the exposure. When light is emitted toward the eye for a long period of time for the purpose of myopia prevention, the irradiance relates to the light emission time as well, and may be increased if the time is short, but is preferably decreased if the time is long. In some embodiments, the irradiance may be 20.0 mW/cm² or less. In some embodiments, the irradiance may be 10.0 mW/cm² or less. In some embodiments, the irradiance may be 80.0 mW/cm² or less. In some embodiments, the irradiance may be 5.0 mW/cm² or less. In some embodiments, the preferred irradiance is 3.0 mW/cm² or less. In some embodiments, the irradiance is preferably 2.0 mW/cm² or less, and is preferably decreased to 1.0 mW/cm², 0.5 mW/cm² or less, 0.1 mW/cm² or less, or 0.05 mW/cm² or less as the time increases. The irradiance may comprise an amount within a range defined by any two of the preceding values. The irradiance can be measured using a known method. It should be noted that “irradiance” indicates the intensity or energy of the light that enters or reaches the eye.

In some embodiments, the structure to contact the eye includes apiece of eyewear such as an eyeglass, or a goggle.

The presently disclosed methods and apparatus are well suited for combination with many types of lenses, such as one or more of: smart contact lenses, contact lenses with antennas and sensors, contact lenses with integrated pulse oximeters, contact lenses with phase map displays, electro-optic contact lenses, contact lenses with flexible conductors, autonomous eye tracking contact lenses, electrochromic contact lenses, dynamic diffractive liquid crystal lenses, automatic accommodation lenses, image display lenses with programmable phase maps, lenses with tear activated micro batteries, tear film sensing contact lenses, lenses with multi-colored LED arrays, contact lenses with capacitive sensing, lenses to detect overlap of an ophthalmic device by an eyelid, lenses with active accommodation, lenses with electrochemical sensors, lenses with enzymes and sensors, lenses including dynamic visual field modulation, lenses for measuring pyruvate, lenses for measuring urea, lenses for measuring glucose, lenses with tear fluid conductivity sensors, lenses with near eye displays with phase maps, or lenses with electrochemical sensor chips.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. (canceled)

2. An apparatus to treat refractive error of an eye, comprising:
a first structure comprising a refractive lens shaped to correct a refractive error of the eye;
a light source coupled to the first structure;
a second structure on the first structure to focus a light energy from the light source toward a retina of the eye and comprising one or more of a refractive, diffractive, or reflective structure; and
a third structure to direct light to the retina of the eye, the third structure configured to be located more anterior to a anterior surface of the eye than the light source and comprising a reflective structure;
wherein the light source and the second structure are configured to concentrate the light energy on the retina of the eye with an irradiance on the retina to treat the refractive error of the eye.

3. The apparatus of claim 1, wherein the light source emits violet light.

4. The apparatus of claim 1, wherein the first structure comprises a contact lens.

5. The apparatus of claim 1, wherein the light energy comprises violet light energy comprising a wavelength within a range from 360 nm to 400 nm.

6. The apparatus of claim 1, wherein the light energy comprises violet light energy and the light source is configured to direct the violet light energy to the retina.

7. The apparatus of claim 1, wherein the light source illuminates a pupil of the eye with the light energy in a range from 0.1 nit to 10 nits.

8. The apparatus of claim 4, wherein the contact lens comprises a lens body, the lens body comprising one or more of a soft contact lens, a hydrogel contact lens, a hard contact lens, a rigid gas permeable contact lens, a polymethyl methacrylate contact lens, or an orthokeratology contact lens.

9. The apparatus of claim 4, wherein the light energy comprises violet light energy and the contact lens is configured to direct the violet light energy onto a cornea and toward the retina with an amount sufficient to promote a curvature change to the cornea of the eye.

10. The apparatus of claim 1, wherein the first structure comprises a contact lens comprising a posterior surface comprising a posterior radius of curvature sized to fit a cornea of the eye, and an anterior surface comprising an anterior radius of curvature configured to correct vision of the eye.

11. The apparatus of claim 1, wherein the structure to contact the eye comprises an implantable structure and wherein the implantable structure comprises a covering disposed over the light source.

12. The apparatus of claim 1, wherein the light source comprises one or more of a radioluminescent light source, a light emitting diode, a laser diode, a radioactive material, or a phosphorescent material.

13. The apparatus of claim 1, wherein the structure to contact the eye comprises an optically transmissive material comprising a transmittance of at least 40% at 360 nm.

14. The apparatus of claim 1, wherein the light source is arranged in a pattern on the structure to contact the eye, the pattern comprising one or more of: a spatial pattern on the structure to contact the eye, a spatial pattern located on an optically used portion of a contact lens, a spatial pattern on an inner portion of the contact lens to transmit light to the retina when a pupil constricts, a circular pattern, or a radial pattern.

15. The apparatus of claim 1, wherein the apparatus comprises a contact lens comprising a posterior surface shaped to receive a cornea of the eye, a contact lens material comprising an index of refraction configured to transmit violet light from the source toward the retina, the contact lens comprising an anterior surface shaped to correct a refractive error of the eye in combination with the index of refraction and the posterior surface.

16. A method, the method comprising treating an eye with the apparatus of claim 1.
